(19) United States
(12) Patent Application Publication
(10) Pub. No.: US 2026/0116866 A1
(43) Pub. Date: Apr. 30, 2026

(54) METHOD FOR PREPARING DITHIOCYCLOPENTENONE DERIVATIVE AND APPLICATION THEREOF

(71) Applicant: INNER MONGOLIA UNIVERSITY OF SCIENCE & TECHNOLOGY, Baotou City (CN)

(72) Inventors: Zhongzhen TIAN, Baotou City (CN); Dongmei Li, Baotou City (CN); Meng YUAN, Baotou City (CN); Mingze PEI, Baotou City (CN)

(21) Appl. No.: 19/220,666

(22) Filed: May 28, 2025

(30) Foreign Application Priority Data
Oct. 30, 2024 (CN) 202411528899.4

(51) Int. Cl. C07D 339/04; A01N 43/26; A01N 43/78; A01P 1/00; C07D 409/12; C07D 417/12

(52) U.S. Cl. CPC C07D 339/04; A01N 43/26; A01N 43/78; A01P 1/00; C07D 409/12; C07D 417/12

(57) ABSTRACT

A method for preparing a dithiocyclopentenone derivative and an application thereof, a bactericide and a bactericidal method are provided. The chemical structural formula of the dithiocyclopentenone derivative is shown as:

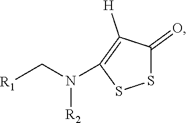

where R₁ is selected from phenyl or substituted phenyl, pyridyl, thiazolyl, thienyl or piperazinyl; and R₂ is selected from H, —CH₃ or —CH₂CH₃.

### CROSS-REFERENCE TO RELATED APPLICATIONS

This application claims priority to Chinese Patent Application No. 202411528899.4, filed on Oct. 30, 2024, the contents of which are hereby incorporated by reference.

### TECHNICAL FIELD

The disclosure belongs to the technical field of organic synthesis, and particularly relates to a method for preparing a dithiocyclopentenone derivative and an application thereof.

### BACKGROUND

Plant diseases seriously threaten the safety and stability of crop production, and effective prevention and control of plant diseases is very important to achieve high crop yield. At present, many bactericides with high efficiency, low toxicity and broad spectrum characteristics are widely used to reduce plant diseases in agriculture. However, due to long-term frequent use, bactericides not only have pesticide residues in agricultural products, but also make plant pathogenic bacteria resistant. Therefore, it is very important for the development of global agriculture to develop bactericides with novel mode of action, low residue and no resistance.

### SUMMARY

Aiming at the above technical problems, the disclosure provides a method for preparing a dithiocyclopentenone derivative and an application thereof.

In order to achieve the above objectives, the present disclosure provides the following technical schemes.

Technical scheme 1: a dithiocyclopentenone derivative, where a chemical structural formula is shown as follows:

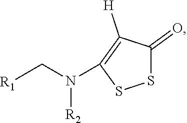

where R₁ is selected from phenyl or substituted phenyl, pyridyl, thiazolyl, thienyl or piperazinyl; and R₂ is selected from H, —CH₃ or —CH₂CH₃.

Technical scheme 2: a method for preparing the dithiocyclopentenone derivative, including following steps:
stirring

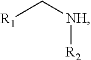

anhydrous potassium carbonate and tetrahydrofuran in an ice water bath, adding dichloro-1,2-dithiocyclopentenone, and reacting at room temperature to obtain an intermediate; and
dissolving the intermediate in acetonitrile, adding iodotrimethylsilane, reacting under a condition of nitrogen at room temperature, stopping the reaction when raw materials are completely reacted, continuously dropping saturated sodium bicarbonate solution and dichloromethane into reaction product for extraction, combining organic phases, and finally stripping once by sodium thiosulfate, drying, concentrating under reduced pressure, and performing column chromatography to obtain the dithiocyclopentenone derivative.

In an embodiment, a molar ratio of the

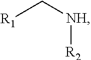

the anhydrous potassium carbonate and the dichloro-1,2-dithiocyclopentenone is 1:2:2; and
a dosage ratio of the

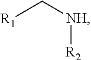

the tetrahydrofuran is 1 gram (g): 40 milliliters (mL).

In an embodiment, a molar ratio of the intermediate to the iodotrimethylsilane is 1:4; and
a dosage ratio of the intermediate to the acetonitrile is 0.3 g: 10 mL.

Technical scheme 3: an application of the dithiocyclopentenone derivative in controlling and killing pathogenic bacteria of plant diseases, where the pathogenic bacteria are pathogenic fungi.

Technical scheme 4: a bactericide, including 1-95 weight percent (wt %) of the dithiocyclopentenone derivative.

In an embodiment, the bactericide further includes agriculturally acceptable surfactants and carriers.

In an embodiment, a dosage form of the bactericide is agriculturally acceptable water dispersible granule, suspension concentrate, granule, dry flowable suspension, emulsifiable concentrate, aqueous solution, oil-based solution, fumigant, aerosol or microcapsule.

Technical scheme 5: an application of the bactericide in controlling and killing pathogenic bacteria of plant diseases, where the pathogenic bacteria are pathogenic fungi.

Technical scheme 6: a bactericidal method, where the bactericide is applied to seeds, plants, fruits or growing soil.

The core innovation of the disclosure lies in that benzene rings and phenyl amine compounds undergo nucleophilic substitution with dichloro-1,2-dithiocyclopentenone to obtain an intermediate, and then the intermediate is dechlorinated by iodosilane to reduce hydrogen, thus obtaining a brand-new dithiocyclopentenone derivative. Dithiocyclopentenone compounds with the same carbon skeleton are constructed by skeleton migration and bioelectronic isosteric arrangement, and hydrogen is introduced into the structure of pharmacodynamic groups. The compound with this structure has excellent antibacterial activity, and has a unique mechanism of action, which makes it difficult to cross-resist other bactericides and has a high antibacterial rate.

Compared with the prior art, the disclosure has the following advantages and technical effects.

The dithiocyclopentenone derivative in the disclosure has high activity to rice blast fungus, and the highest antibacterial rate to rice blast fungus may reach 97.9% at the concentration of 100 parts per million (ppm).

The dithiocyclopentenone derivative in the disclosure may be used to prepare bactericides in various dosage forms for controlling and eliminating pathogenic fungi of plant diseases.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. The intermediate value within any stated value or stated range and every smaller range between any other stated value or intermediate value within the stated range are also included in the present disclosure. The upper and lower limits of these smaller ranges may be independently included or excluded from the range.

Unless otherwise specified, all technical and scientific terms used herein have the same meaning as commonly understood by one of ordinary skill in the art to which this disclosure relates. Although the present disclosure only describes the preferred methods and materials, any methods and materials similar or equivalent to those described herein may also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the disclosure. The description and embodiments of that present disclosure are exemplary only.

The terms “including”, “comprising”, “having” and “containing” used in this article are all open terms, which means including but not limited to.

The term “active substance of the present disclosure” or “active compound of the present disclosure” refers to dithiocyclopentenone derivatives of the present disclosure, which have high antibacterial activity.

A dithiocyclopentenone derivative, where a chemical structural formula is shown as follows:

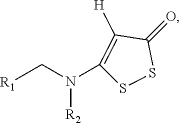

where R₁ is selected from phenyl or substituted phenyl, pyridyl, thiazolyl, thienyl or piperazinyl; and R₂ is selected from H, —CH₃ or —CH₂CH₃.

A method for preparing the dithiocyclopentenone derivative includes the following chemical synthesis methods:

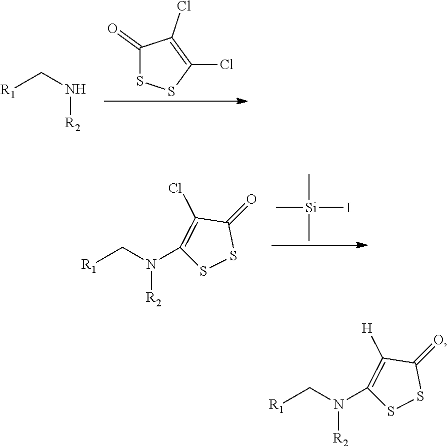

and specifically includes the following steps:
(1) stirring

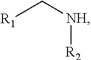

anhydrous potassium carbonate and tetrahydrofuran in an ice water bath, adding dichloro-1,2-dithiocyclopentenone, and reacting at room temperature to obtain an intermediate; and
(2) dissolving the intermediate in acetonitrile, adding iodotrimethylsilane, reacting under a condition of nitrogen at room temperature, stopping the reaction when raw materials are completely reacted, continuously dropping saturated sodium bicarbonate solution and dichloromethane into reaction product for extraction, combining organic phases, and finally stripping once by sodium thiosulfate, drying, concentrating under reduced pressure, and performing column chromatography to obtain the dithiocyclopentenone derivative.

In the following preferred embodiments of the present disclosure, a molar ratio of the

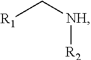

the anhydrous potassium carbonate and the dichloro-1,2-dithiocyclopentenone is 1:2:2; a dosage ratio of the

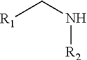

to the tetrahydrofuran is 1 g: 40 mL; a molar ratio of the intermediate to the iodotrimethylsilane is 1:4; and a dosage ratio of the intermediate to the acetonitrile is 0.3 g: 10 mL.

An application of the dithiocyclopentenone derivative in controlling and killing pathogenic bacteria of plant diseases, where the pathogenic bacteria are pathogenic fungi.

The active substance of the disclosure may be used for controlling and killing pathogenic fungi causing plant diseases, and may be used for crop protection, where the crops are cereals, corn, rice, soybeans and other leguminous plants, fruits and fruit trees, nuts and nut trees, oranges and citrus trees, any horticultural plants, cucurbitaceae plants, oil plants, tobacco, coffee, tea, cocoa, beets, sugarcane, cotton, potatoes, tomatoes, onions, peppers and other vegetables and ornamental plants. The term “pathogenic bacteria” in this specification refers to pathogenic fungi that are plant diseases. Specifically, for example, the following plant diseases caused by pathogenic bacteria may be cited, but the specific examples are not limited to this.

Examples include Pricuaria oryzae, Helminthosporium oryzae, Erysiphe graminis, Fusarium graminearum, Septoria tritici, Phomaci tricarpa, Colletrichum gloeosporioides, Sclerotinia mali, Podosphaera leucotricha, Alternaria mali, Vnturia pirina, Gymnosporangium haraenum, Gloeosporium laelicolor, Gloeosporium laeticolor, Plasmopara viticola, Sphaceloma ampelinum, Glomerella cingulala, Pseudoperonospora cubensis, Colletotrichum lagenarium, Sphaerotheca fuliginea, Mycosphaerella melonis, Botrytis cinerea, Altermaria solani, Cladosporium fiuvum, Phytophthorain festans, Alternaria brassicae, Ceroosporella brassicae, Cilletotrichum boehmieriae, Mycosphaerella personatu, Cercospora arachidicola, Phytophthora infestans, Sphaerotheca humuli, Erysiphe cichoraoearm, Colletotrichum nicotianae, Cercospora beticola, Diplocarpon rosae, Sphaerotheca pannosa, Cercospora vicosae, Colletotrichum gloeosporioides, Peronophthora litchic, Cercospora musae, Gioeosporium musarum, Colletotrichum gloeosporioides; Colletotrichum capsici, Colletotrichum gloeosporioides and other plant diseases.

As a disease for which the application of the bactericide of the present disclosure is more optional, Pricuaria oryzae of rice may be cited.

A bactericide prepared from the dithiocyclopentenone derivative includes 1-95 wt % of dithiocyclopentenone derivative as an active ingredient, and the rest are agriculturally acceptable adjuvants and carriers. The adjuvants include surfactants, adhesives, dispersants, stabilizers and other preparations.

A dosage form of the bactericide is agriculturally acceptable water dispersible granule, suspension concentrate, granule, dry flowable suspension, emulsifiable concentrate, aqueous solution, oil-based solution, fumigant, aerosol or microcapsule. These preparations may be prepared by general methods. For example, the active substance is mixed with a liquid solvent and/or a solid carrier, and at the same time, a surfactant, an emulsifier, a dispersant, a stabilizer and a wetting agent are added, and an adhesive, a dye or a flavoring agent may also be added.

As the surfactant, nonionic surfactants such as polyoxyethylene alkyl ether, polyoxyethylene polyoxypropylene block copolymer, polyoxyethylene fatty acid (mono or di) ester, sorbitan fatty acid ester, castor oil ethylene oxide adduct, acetylene glycol, acetylene alcohol, ethylene oxide adduct of acetylene glycol, alkyl glycoside, etc.; anion surfactant such as alkyl sulfate, alkyl benzene sulfonate, alkyl sulfonated succinate, naphthalene sulfonate, formalin condensate of naphthalene sulfonic acid, polyoxyethylene alkyl ether sulfate or phosphate, polyoxyethylene (mono- or di-) alkylphenyl ether sulfate or phosphate, polycarboxylic acid salt (such as polyacrylate, polymaleate and copolymer of maleic acid and olefin, etc.) and polystyrene sulfonate; cationic surfactants such as alkyl amine salts and alkyl quaternary ammonium salts; amphoteric surfactants such as amino acid type and betaine type; and silicone surfactants, fluorine surfactants, etc. may be selected.

As the adhesive or dispersant, casein, gelatin, various (starch, Arabic resin, fiber derivatives, etc.), lignin derivatives, bentonite, sugars, synthetic water-soluble polymers, etc. may be selected.

As stabilizers, such as acidic isopropyl phosphate vegetable oil, mineral oil, fatty acid, etc. may be selected.

The carrier may be a liquid carrier and/or a gas carrier. Suitable solid carriers include natural and synthetic clays and silicates, such as natural silica and diatomaceous earth; magnesium silicate, such as talc; magnesium aluminum silicate, such as active surface soil and vermiculite; aluminum silicate such as kaolinite, montmorillonite, mica; calcium carbonate, calcium sulfate and ammonium sulfate; synthetic hydrated silica and synthetic calcium silicate or aluminum silicate. Solid carriers especially suitable for powder include naturally occurring rock powders, such as attapulgite, bentonite, montmorillonite and diatomite; synthetic ground minerals, such as finely dispersed silicic acid or alumina. Carriers particularly suitable for granules include crushed and graded natural rocks, such as calcite, marble, pumice, sepiolite, dolomite and synthetic particles made of organic and inorganic powders. In addition, particles may be prepared from organic materials, such as sawdust, peanut shells, corn cob fibers and dried tobacco stalks.

Suitable liquid carriers include water: water may be used as solvent or diluent, and organic solvents may also be used as auxiliary solvents or antifreeze additives. Suitable organic solvents include aromatic hydrocarbons, such as toluene, xylene, benzene, phenylalkylnaphthalene and chlorinated aromatic hydrocarbons; chlorinated aliphatic hydrocarbons such as chlorobenzene, vinyl chloride, trichloroethane, dichloromethane, chloroform, carbon tetrachloride and polychloroethane; aliphatic hydrocarbons such as petroleum fractions, cyclohexane, light mineral oil and paraffin wax; however, polar solvents, namely alcohols, such as isopropanol, butanol, ethylene glycol, benzyl alcohol, furfuryl alcohol, cyclohexanol, ether and ester, are particularly suitable; ketones such as acetone, methyl ethyl ketone, methyl ethyl ketone, methyl isobutyl ketone, cyclohexanone and butyrolactone, dimethylformamide (DMF), dimethyl sulfoxide and N-methylpyrrolidone. Mixtures of different liquids are often suitable.

These solid and liquid carriers may be used alone or in combination of two or more.

By adding one or more other bactericides to the composition, it may have a broader spectrum of activity than the single compound of general formula. In addition, other bactericides may have synergistic effect on the antibacterial activity of the compound of general formula. The compound of the general formula may also be mixed with other pesticides, or mixed with another bactericide and other pesticides at the same time. Because the active compound itself has activity, it may also be used alone.

The amount of effective components in the bactericide of the present disclosure is usually optionally 1-90 wt % relative to the whole preparation. When the active ingredient of the present disclosure is used as a bactericide, the application rate may vary within a relatively wide range, depending on the type of application. The application rate of the active ingredients of the present disclosure is: when treating plant parts, such as leaves: 0.1-10000 grams per hectare (g/ha), optionally 10-1000 g/ha, more optionally 50-300 g/ha (when applying by irrigation or drip irrigation, the application rate may even be reduced, especially when using inert substances such as rock wool or perlite); and when treating seeds: 2-200 g per 100 kilograms (kg) of seeds, optionally 2.5-150 g per 100 kg of seeds, more optionally 2.5-25 g per 100 kg of seeds, even more optionally 2.5-12.5 g per 100 kg of seeds; and when treating soil: 0.1-10000 g/ha, optionally 1-5000 g/ha.

The method for preparing bactericides with different dosage forms by using the dithiocyclopentenone derivatives of the disclosure may be a conventional method, such as:
(1) wettable powder bactericide: the following components are prepared in proportion: 30% (weight percentage, the same below) of any compound in the compound I series; 2% sodium dodecyl sulfate; 3% sodium lignosulfonate; 5% naphthalene sulfonic acid formaldehyde condensate and 60% light calcium carbonate. The components are fully mixed and crushed by an ultra-fine pulverizer to obtain 30% wettable powder bactericide;
(2) suspension concentrate bactericide: the following components are prepared in proportion: 40% of any compound in the compound I series; 10% ethylene glycol; 6% nonylphenol polyethylene glycol ether; 5% sodium lignosulfonate; 1% carboxymethyl cellulose; 0.2% aqueous formaldehyde solution (37%); 0.8% silicone oil water emulsion water (75%) and 37% water. The components are fully mixed to obtain 40% suspension concentrate bactericide; and
(3) water dispersible granule bactericide: the following components are prepared in proportion: 60% of any compound in the compound I series; 12% naphthalene sulfonic acid formaldehyde condensate; 8% N-methyl-N-oleoyl-sodium taurate; 2% polyvinylpyrrolidone; 2% carboxymethyl cellulose and 16% kaolin. All components are mixed and then crushed. Water is added and kneaded into the mixture. The mixture is then fed into a granulator equipped with a 10-100 mesh sieve for granulation. The granules are subsequently dried and sieved to obtain a 60% water dispersible granule bactericide.

The wettable powder and water dispersible granules thus obtained may be used by diluting them with water to a specified concentration, making them into solutions, suspensions or emulsions, and then spreading them on plants.

The application of the bactericide prepared by the disclosure in controlling and eliminating pathogenic bacteria of plant diseases, where the pathogenic bacteria are pathogenic fungi.

The bactericidal method refers to applying the bactericide to seeds, plants, fruits or growing soil.

Unless otherwise specified, “room temperature” in the present disclosure refers to 20-30 degrees Celsius (° C.).

The raw materials used in the disclosure are all purchased in the market.

The technical scheme of the present disclosure will be further explained by embodiments.

The synthetic route is as follows:

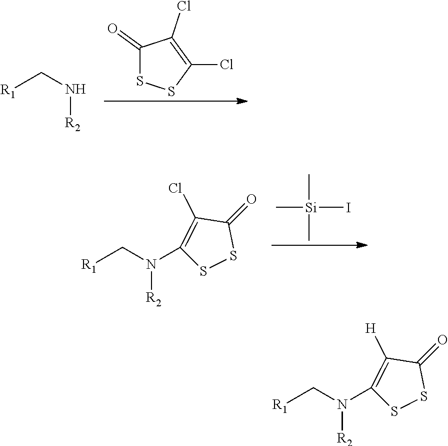

### Embodiment 1

### Synthesis of Compound I1 (R₁=3-F-phenyl; R₂=H)

3-fluoromethylamine (1 g, 7.99 millimoles (mmol)), anhydrous potassium carbonate (2.21 g, 15.98 mmol) and 40 mL of dry tetrahydrofuran are added into a 150 mL round-bottomed flask and are stirred in an ice water bath for 10 minutes (min). Then dichloro-1,2-dithiocyclopentenone (2.99 g, 15.99 mmol) is slowly added into the mixed solution, and the ice is removed. The reaction is allowed to proceed at room temperature. The reaction progress is monitored by Thin-Layer Chromatography (TLC). After the raw materials are completely consumed, the reaction is stopped. The mixed solution is filtered under vacuum. The filtrate is concentrated under reduced pressure by distillation. The mixed solution is purified by column chromatography (V (dichloromethane): V (methanol)=5:1) to afford 0.5 g of the intermediate 4-chloro-5-((3-fluorobenzyl) amino)-3H-1,2-dithiol-3-one.

The intermediate (0.30 g, 1.09 mmol) is dissolved in 10 mL of acetonitrile and transferred into a 50 mL three-necked flask. Iodotrimethylsilane (0.87 g, 4.35 mmol) is added to the solution. The reaction is carried out at room temperature under nitrogen atmosphere, and the progress is monitored by TLC. After complete consumption of the raw materials are confirmed, the reaction is quenched. A saturated sodium bicarbonate solution (10 mL) is slowly added dropwise to the mixed solution. The mixed solution is extracted with 20 mL of dichloromethane each time, and the extraction is performed three times. The organic phases are combined. The combined organic layer is back-extracted once with sodium thiosulfate, dried over anhydrous sodium sulfate, and concentrated under reduced pressure. The crude product is purified by column chromatography (petroleum ether: ethyl acetate=3:1) to afford the target compound as a brown oil with a yield of 76.9%. ¹H NMR (400 megahertz (MHz), CDCl₃) δ 7.59 (singlet (s), —NH, 1 hydrogen (H)), 7.33 (triplet, coupling constant (t, J)=7.5 hertz (Hz), Ph—H, 1H), 7.24 (doublet of triplets, coupling constant (dt, J)=7.5, 1.1 Hz, Ph—H, 1H), 7.03 (dt, J=7.3, 2.0 Hz, Ph—H, 1H), 6.98-6.96 (multiplet (m), Ph—H, 1H), 5.59 (s, ═CH—, 1H), 4.53 (s, Ph—CH₂—N, 2H); ¹³C NMR (101 MHz, CDCl₃) δ 189.46, 168.56, 164.91, 138.03, 132.19, 128.60, 118.02, 117.84, 112.58, 48.15.

### Embodiment 2

### Synthesis of Compound I2 (R₁=phenyl; R₂=H)

The synthetic method is the same as that in Embodiment 1, only the substituents are different, and the target compound is obtained as a brown oil with a yield of 80.8%. ¹H NMR (400 MHz, CDCl₃) δ 7.42 (s, —NH, 1H), 7.32 (doublet, coupling constant (d, J)=0.8 Hz, Ph—H, 2H), 7.31-7.28 (m, Ph—H, 2H), 7.27-7.23 (m, Ph—H, 1H), 5.59 (s, ═CH—, 1H), 4.49 (s, Ph—CH₂—N, 2H). ¹³C NMR (101 MHz) δ 189.46, 166.61, 143.58, 132.39, 131.28, 131.12, 112.58, 52.08.

### Embodiment 3

### Synthesis of Compound I3 (R₁=3-Cl-phenyl; R₂=H)

The synthetic method is the same as that in Embodiment 1, only the substituents are different, and the target compound is obtained as the yellow solid with a yield of 72.7%. mp=117.5-118.6° C. ¹H NMR (400 MHz, CDCl₃) δ 7.59 (s, —NH—, 1H), 7.30-7.23 (m, Ph—H, 4H), 5.59 (s, ═CH—, 1H), 4.58 (s, Ph—CH₂—N, 2H); ¹³C NMR (101 MHz, CDCl₃) δ 189.46, 167.79, 139.13, 135.95, 132.28, 131.74, 131.07, 130.84, 112.58, 51.33. HRMS (ESI+): calcd for C₁₀H₉ClNO₂S₂(M+H)⁺ 257.9808, found, 257.9809.

### Embodiment 4

### Synthesis of Compound I4 (R₁=3-CN-phenyl; R₂=H)

The synthetic method is the same as that in Embodiment 1, only the substituents are different, and the target compound is obtained as light yellow powder with a yield of 69.7%. mp=69.2-70.1° C. ¹H NMR (400 MHz, CDCl₃) δ 7.59 (s, —NH, 1H), 7.67 (d, J=1.1 Hz, Ph—H, 1H), 7.56-7.52 (m, Ph—H, 1H), 7.35-7.30 (m, Ph—H, 2H), 5.59 (s, ═CH—, 1H), 4.51 (s, Ph—CH₂—N, 2H); ¹³C NMR (101 MHz, CDCl₃) δ 189.46, 167.79, 138.67, 136.11, 133.83, 132.00, 130.64, 120.11, 113.18, 112.58, 51.65.

### Embodiment 5

### Synthesis of Compound I5 (R₁=3,5—(OCH₃)₂-phenyl; R₂=H)

The synthetic method is the same as that in Embodiment 1, only the substituents are different, and the target compound is obtained as beige white powder with a yield of 40.3%. mp=130.9-131.1° C. ¹H NMR (400 MHz, CDCl₃) δ 7.54 (s, —NH, 1H), 6.66 (dt, J=2.0, 1.0 Hz, Ph—H, 2H), 6.41 (t, J=2.0 Hz, Ph—H, 1H), 5.59 (s, ═CH—, 1H), 4.44 (s, Ph—CH₂—N, 2H), 3.81 (s, Ph-OCH₃, 6H); ¹³C NMR (101 MHz, CDCl₃) δ 189.46, 168.08, 161.91, 138.49, 112.58, 108.99, 100.51, 56.17, 48.68.

### Embodiment 6

### Synthesis of Compound I6 (R₁=4,5—(OCH₃)₂-phenyl; R₂=H)

The synthetic method is the same as that in Embodiment 1, only the substituents are different, and the target compound is obtained as a light yellow solid with a yield of 72.9%. mp=101.0-101.5° C. ¹H NMR (400 MHz, CDCl₃) δ 7.60 (s, —NH, 1H), 6.86 (d, J=1.0 Hz, Ph—H, 1H), 6.84 (d, J=7.4 Hz, Ph—H, 1H), 6.82-6.79 (m, Ph—H, 1H), 5.59 (s, ═CH—, 1H), 4.48 (s, Ph—CH₂—N, 2H), 3.85 (s, Ph-OCH₃, 3H), 3.83 (s, Ph-OCH₃, 3H); ¹³C NMR (101 MHz, CDCl₃) δ 189.46, 168.98, 150.53, 150.23, 132.37, 123.58, 112.58, 112.26, 112.25, 56.50, 56.49, 50.25.

### Embodiment 7

### Synthesis of Compound I7 (R₁=2,4—(OCH₃)₂-phenyl; R₂=H)

The synthetic method is the same as that in Embodiment 1, only the substituents are different, and the target compound is obtained as an orange solid with a yield of 40.8%. mp=84.6-85.1° C. ¹H NMR (400 MHz, CDCl₃) δ 7.45 (s, —NH, 1H), 7.16 (dt, J=7.5, 1.0 Hz, Ph—H, 1H), 6.68 (doublet of doublets, coupling constant (dd, J)=7.5, 2.0 Hz, Ph—H, 1H), 6.44 (d, J=2.0 Hz, Ph—H, 1H), 5.59 (s, ═CH—, 1H), 4.58 (s, Ph—CH₂—N, 2H), 3.83 (s, Ph-OCH₃, 3H), 3.80 (s, Ph-OCH₃, 3H); ¹³C NMR (101 MHz, CDCl₃) δ 189.46, 165.47, 162.51, 161.68, 132.45, 121.87, 111.71, 107.75, 99.41, 56.29, 56.24, 50.02. HRMS (ESI+): calcd for C₁₂H₁₄NO₃S₂(M+H)⁺ 284.0409, found, 284.0410.

### Embodiment 8

### Synthesis of Compound I8 (R₁=2,3—(OCH₃)₂-phenyl; R₂=H)

The synthetic method is the same as that in Embodiment 1, only the substituents are different, and the target compound is obtained as light yellow powder with a yield of 83.3%. mp=137.4-137.9° C. ¹H NMR (400 MHz, CDCl₃) δ 7.53 (s, —NH, 1H), 7.01-6.96 (m, Ph—H, 1H), 6.88 (t, J=7.5 Hz, Ph—H, 1H), 6.82 (dd, J=7.5, 2.0 Hz, Ph—H, 1H), 5.59 (s, ═CH—, 1H), 4.48 (s, Ph-CH₂—N, 2H), 3.84 (s, Ph-OCH₃, 3H), 3.84 (s, Ph-OCH₃, 3H); ¹³C NMR (101 MHz, CDCl₃) δ 189.46, 164.13, 150.23, 150.01, 128.73, 124.25, 123.76, 114.02, 111.71, 61.24, 56.53, 48.97.

### Embodiment 9

### Synthesis of Compound I9 (R₁=4—F-phenyl; R₂=H)

The synthetic method is the same as that in Embodiment 1, only the substituents are different, and the target compound is obtained as orange powder with a yield of 67.4%. mp=139.1-140.5° C. ¹H NMR (400 MHz, CDCl₃) δ 7.42 (s, —NH, 1H), 7.31 (dt, J=7.5, 1.1 Hz, Ph—H, 2H), 7.08-7.05 (m, Ph—H, 2H), 5.59 (s, ═CH—, 1H), 4.46 (s, Ph—CH₂—N, 2H); ¹³C NMR (101 MHz, CDCl₃) δ 189.46, 166.61, 164.46, 137.76, 132.22, 132.22, 116.97, 116.97, 112.58, 51.72.

### Embodiment 10

### Synthesis of Compound I10 (R₁=3—OCH₃-phenyl; R₂=H)

The synthetic method is the same as that in Embodiment 1, only the substituents are different, and the target compound is obtained as beige white powder with a yield of 76.9%. mp=96.7-97.3° C. ¹H NMR (400 MHz, CDCl₃) δ 7.68 (s, —NH—, 1H), 7.25 (t, J=7.7 Hz, Ph—H, 1H), 7.19 (doublet of triplets of triplets (dtt, J)=7.5, 1.9, 1.0 Hz, Ph—H, 1H), 6.87 (d, J=2.0 Hz, Ph—H, 1H), 6.86 (quartet, coupling constant (q, J)=2.0 Hz, Ph—H, 1H), 5.59 (s, ═CH—, 1H), 4.46 (s, Ph-CH₂—N, 2H), 3.80 (s, —OCH₃, 3H); ¹³C NMR (101 MHz, CDCl₃) δ 189.46, 168.56, 161.36, 139.52, 131.44, 125.50, 116.27, 113.40, 112.58, 56.01, 49.96. HRMS (ESI+): calcd for C₁₁H₁₂NO₂S₂(M+H)⁺ 254.0302, found, 254.0304.

### Embodiment 11

### Synthesis of Compound I11 (R₁=5,6—F-phenyl; R₂=H)

The synthetic method is the same as that in Embodiment 1, only the substituents are different, and the target compound is obtained as an orange solid with a yield of 90.5%. mp=96.3-97.0° C. ¹H NMR (400 MHz, CDCl₃) δ 7.42 (s, —NH, 1H), 7.23 (s, Ph—H, 1H), 7.22 (s, Ph—H, 1H), 7.17-7.13 (m, Ph—H, 1H), 5.59 (s, ═CH—, 1H), 4.38 (s, Ph—CH₂—N, 2H); ¹⁹F NMR (565 MHz, CDCl₃) δ −136.95 (d, J=20.9 Hz, 1 fluorine atom (F)), −142.82 (d, J=20.3 Hz, 1F). ¹³C NMR (101 MHz, CDCl₃) δ 189.46, 164.86, 151.66, 150.66, 128.48, 127.84, 123.82, 117.24, 111.71, 50.13. HRMS (ESI+): calcd for C₁₀H₈F₂NOS₂(M+H)⁺ 260.0009, found, 260.0010.

### Embodiment 12

### Synthesis of Compound I12 (R₁=3,6—Cl-phenyl; R₂=H)

The synthetic method is the same as that in Embodiment 1, only the substituents are different, and the target compound is obtained as the orange powder with a yield of 78.1%. mp=86.7-87.9° C. ¹H NMR (400 MHz, CDCl₃) δ 7.40 (s, —NH, 1H), 7.38-7.35 (m, Ph—H, 1H), 7.25 (d, J=7.4 Hz, Ph—H, 1H), 7.21 (dd, J=7.5, 2.0 Hz, Ph—H, 1H), 5.59 (s, ═CH—, 1H), 4.54 (s, Ph—CH₂—N, 2H); ¹³C NMR (101 MHz, CDCl₃) δ 189.46, 167.17, 135.27, 134.87, 133.24, 132.14, 131.42, 130.82, 111.45, 49.82.

### Embodiment 13

### Synthesis of Compound I13 (R₁=4—N(CH₃)₂-phenyl; R₂=H)

The synthetic method is the same as that in Embodiment 1, only the substituents are different, and the target compound is obtained as brownish yellow powder with a yield of 52.0%. mp=136.0-136.5° C. ¹H NMR (400 MHz, CDCl₃) δ 7.42 (s, —NH, 1H), 7.21 (t, J=1.0 Hz, Ph—H, 1H), 7.20 (t, J=1.1 Hz, Ph—H, 1H), 6.71 (d, J=1.6 Hz, Ph—H, 1H), 6.70 (d, J=1.6 Hz, Ph—H, 1H), 5.59 (s, ═CH—, 1H), 4.45 (s, Ph—CH₂—N, 2H), 2.95 (s, Ph-N(CH₃)₂, 6H); ¹³C NMR (101 MHz, CDCl₃) δ 189.46, 166.61, 152.01, 137.45, 131.36, 115.26, 112.58, 51.74, 41.19.

### Embodiment 14

### Synthesis of Compound I14 (R₁=4—OCH₃-phenyl; R₂=H)

The synthetic method is the same as that in Embodiment 1, only the substituents are different, and the target compound is obtained as beige white solid with a yield of 76.9%. mp=158.1-158.3° C. ¹H NMR (400 MHz, CDCl₃) δ 7.42 (s, —NH, 1H), 7.20 (t, J=1.0 Hz, Ph—H, 1H), 7.18 (t, J=1.0 Hz, Ph—H, 1H), 6.89 (d, J=1.6 Hz, Ph—H, 1H), 6.88 (d, J=1.5 Hz, Ph—H, 1H), 5.59 (s, ═CH—, 1H), 4.46 (s, Ph—CH₂—N, 2H), 3.79 (s, Ph-OCH₃, 3H); ¹³C NMR (101 MHz, CDCl₃) δ 189.46, 166.61, 161.44, 136.01, 131.47, 131.47, 115.68, 115.68, 112.58, 56.03, 51.72. HRMS (ESI+): calcd for C₁₁H₁₂NO₂S₂(M+H)⁺ 254.0302, found, 254.0304.

### Embodiment 15

### Synthesis of Compound I15 (R₁=3—CN-phenyl; R₂=—CH₃)

The synthetic method is the same as that in Embodiment 1, only the substituents are different, and the target compound is obtained as beige solid with a yield of 57.7%. mp=69.2-71.9° C. ¹H NMR (400 MHz, CDCl₃) δ 7.54 (dt, J=7.5, 2.0 Hz, Ph—H, 1H), 7.49 (doublet of pentets, coupling constant (dp, J)=1.9, 0.9 Hz, Ph—H, 1H), 7.36 (d, J=14.9 Hz, Ph—H, 1H), 7.27 (d, J=7.5 Hz, Ph—H, 1H), 5.58 (s, ═CH—, 1H), 4.51 (t, J=1.0 Hz, Ph—CH₂—N, 2H), 3.15 (s, —CH₃, 3H); ¹³C NMR (101 MHz, CDCl₃) δ 189.50, 162.98, 138.74, 136.14, 133.83, 131.56, 130.70, 120.11, 113.54, 108.98, 58.38, 40.98.

### Embodiment 16

### Synthesis of Compound I16 (R₁=phenyl; R₂=—CH₃)

The synthetic method is the same as that in Embodiment 1, only the substituents are different, and the target compound is obtained as a light yellow solid with a yield of 88.0%. mp=103.6-104.2° C. ¹H NMR (400 MHz, CDCl₃) δ 7.43-7.32 (m, Ph—H, 3H), 7.26-7.20 (m, Ph—H, 2H), 5.57 (s, ═CH—, 1H), 4.61 (s, Ph—CH₂—N, 2H), 3.11 (s, —CH₃, 3H). ¹³C NMR (101 MHz, CDCl₃) δ 189.22, 173.86, 134.62, 129.16, 128.39, 127.12, 93.08, 58.03, 39.87.

### Embodiment 17

### Synthesis of Compound I17 (R₁=3—Cl-phenyl; R₂=—CH₃)

The synthetic method is the same as that in Embodiment 1, only the substituents are different, and the target compound is obtained as beige white solid with a yield of 92.3%. mp=72.6-73.3° C. ¹H NMR (400 MHz, CDCl₃) δ 7.33 (dd, J=2.6, 1.2 Hz, Ph—H, 1H), 7.32 (d, J=1.3 Hz, Ph—H, 1H), 7.21 (dt, J=2.5, 1.1 Hz, Ph—H, 1H), 7.12 (doublet of triplets of doublets, coupling constant (dtd, J)=5.2, 1.8, 0.9 Hz, Ph—H, 1H), 5.55 (s, ═CH—, 1H), 4.57 (s, Ph—CH₂—N, 2H), 3.12 (s, —CH₃, 3H). ¹³C NMR (101 MHz, CDCl₃) δ 189.17, 173.76, 136.79, 135.11, 130.50, 128.60, 127.11, 125.16, 93.38, 57.47, 40.04.

### Embodiment 18

### Synthesis of Compound I18 (R₁=3—F-phenyl; R₂=—CH₃)

The synthetic method is the same as that in Embodiment 1, only the substituents are different, and the target compound is obtained as a brown solid with a yield of 76.9%. mp=70.9-72.7° C. ¹H NMR (400 MHz, CDCl₃) δ 7.40-7.34 (m, Ph—H, 1H), 7.06-7.00 (m, Ph—H, 2H), 6.93 (dt, J=9.4, 2.1 Hz, Ph—H, 1H), 5.55 (s, ═CH—, 1H), 4.59 (s, Ph—CH₂—N, 2H), 3.13 (s, —CH₃, 3H). ¹³C NMR (101 MHz, CDCl₃) δ 189.16, 164.42, 161.95, 137.30, 137.23, 130.90, 130.82, 122.63, 122.60, 115.46, 115.25, 114.08, 113.86, 93.34, 57.52, 57.50, 40.04. HRMS (ESI+): calcd for C₁₁H₁₁FNOS₂(M+H)⁺ 256.0260, found, 256.0261.

### Embodiment 19

### Synthesis of Compound I19 (R₁=4—F-phenyl; R₂=—CH₃)

The synthetic method is the same as that in Embodiment 1, only the substituents are different, and the target compound is obtained as a yellow solid with a yield of 80.7%. mp=56.6-57.8° C. ¹H NMR (400 MHz, CDCl₃) δ 7.19 (dd, J=8.4, 5.2 Hz, Ph—H, 2H), 7.06 (t, J=8.6 Hz, Ph—H, 2H), 5.54 (s, ═CH—, 1H), 4.55 (s, Ph—CH₂—N, 2H), 3.07 (s, —CH₃, 3H). ¹³C NMR (101 MHz, CDCl₃) δ 189.35, 163.94, 161.48, 130.47, 129.05, 128.97, 116.36, 116.15, 93.32, 57.40, 39.84. HRMS (ESI+): calcd for C₁₁H₁₁FNOS₂(M+H)⁺ 256.0259, found, 256.0261.

### Embodiment 20

### Synthesis of Compound I20 (R₁=3—OCH₃-phenyl; R₂=—CH₃)

The synthetic method is the same as that in Embodiment 1, only the substituents are different, and the target compound is obtained as a light yellow powder with a yield of 90.9%. mp=106.3-107.8° C. ¹H NMR (400 MHz, CDCl₃) δ 7.28 (d, J=7.9 Hz, Ph—H, 1H), 6.85 (dd, J=8.3, 2.5 Hz, Ph—H, 1H), 6.81-6.77 (m, Ph—H, 1H), 6.73 (t, J=2.1 Hz, Ph—H, 1H), 5.55 (s, ═CH—, 1H), 4.55 (s, Ph—CH₂—N, 2H), 3.80 (s, —OCH₃, 3H), 3.09 (s, —CH₃, 3H). ¹³C NMR (101 MHz, CDCl₃) δ 189.40, 173.94, 160.28, 136.30, 130.38, 119.33, 113.35, 113.13, 93.25, 58.04, 55.43, 40.02. HRMS (ESI+): calcd for C₁₂H₁₄FNO₂S₂(M+H)⁺ 268.0458, found, 268.0460.

### Embodiment 21

### Synthesis of Compound I21 (R₁=4—OCH₃-phenyl; R₂=—CH₃)

The synthetic method is the same as that in Embodiment 1, only the substituents are different, and the target compound is obtained as light yellow oil with a yield of 51.9%. ¹H NMR (400 MHz, CDCl₃) δ 7.17 (d, J=6.6 Hz, Ph—H, 1H), 7.13 (d, J=2.1 Hz, Ph—H, 1H), 6.93-6.91 (m, Ph—H, 1H), 6.84-6.81 (m, Ph—H, 1H), 5.56 (s, ═CH—, 1H), 4.53 (s, Ph—CH₂—N—, 2H), 3.83 (s, —OCH₃, 3H), 3.06 (s, —CH₃, 3H). ¹³C NMR (101 MHz, CDCl₃) δ 189.32, 159.68, 158.38, 128.99, 128.68, 126.49, 114.51, 114.21, 92.93, 57.52, 55.38, 55.29, 39.56. HRMS (ESI+): calcd for C₁₂H₁₄FNO₂S₂(M+H)⁺ 268.0457, found, 268.0460.

### Embodiment 22

### Synthesis of Compound I22 (R₁=3,5—(OCH₃)₂-phenyl; R₂=—CH₃)

The synthetic method is the same as that in Embodiment 1, only the substituents are different, and the target compound is obtained as a light yellow solid with a yield of 76.9%. mp=140.4-140.8° C. ¹H NMR (400 MHz, CDCl₃) δ 6.66 (dt, J=2.0, 0.9 Hz, Ph—H, 2H), 6.41 (t, J=2.0 Hz, Ph—H, 1H), 5.56 (s, ═CH—, 1H), 4.47 (s, Ph—CH₂—N, 2H), 3.81 (s, Ph-OCH₃, 6H), 3.15 (s, —CH₃, 3H); ¹³C NMR (101 MHz, CDCl₃) δ 189.50, 162.23, 161.30, 161.30, 135.42, 108.98, 108.51, 108.51, 100.68, 56.17, 56.17, 55.59, 40.65.

### Embodiment 23

### Synthesis of Compound I23 (R₁=3—F-phenyl; R₂=—CH₂CH₃)

The synthetic method is the same as that in Embodiment 1, only the substituents are different, and the target compound is obtained as yellow oil with a yield of 83%. ¹H NMR (400 MHz, CDCl₃) δ 7.37-7.31 (m, Ph—H, 1H), 7.03-6.91 (m, Ph—H, 3H), 5.52 (s, ═CH—, 1H), 4.57 (s, Ph—CH₂—N, 2H), 3.48 (q, J=7.1 Hz, N—CH₂—, 2H), 1.25 (t, J=7.2 Hz, —CH₃, 3H). ¹³C NMR (101 MHz, CDCl₃) δ 189.47, 173.09, 164.52, 162.06, 137.61, 130.88, 122.55, 114.63, 93.37, 55.23, 48.10, 12.22. HRMS (ESI+): calcd for C₁₂H₁₃FNOS₂(M+H)⁺ 270.0421, found, 270.0417.

### Embodiment 24

### Synthesis of Compound I24 (R₁=3—OCF₃-phenyl; R₂=H)

The synthetic method is the same as that in Embodiment 1, only the substituents are different, and the target compound is obtained as an orange solid with a yield of 76.9%. mp=75.6-76.8° C. ¹H NMR (400 MHz, CDCl₃) δ 7.42 (d, J=7.9 Hz, —NH, 1H), 7.25 (s, Ph—H, 1H), 7.24 (s, Ph—H, 1H), 7.19 (d, J=8.3 Hz, Ph—H, 1H), 7.16 (s, Ph—H, 1H), 5.58 (s, ═CH—, 1H), 4.44 (d, J=4.2 Hz, Ph—CH₂—N, 2H). ¹³C NMR (101 MHz, CDCl₃) δ 190.40, 173.29, 149.81, 138.13, 130.66, 125.84, 121.76, 120.80, 120.08, 94.30, 49.88.

### Embodiment 25

### Synthesis of Compound I25 (R₁=thiophenyl; R₂=—CH₃)

The synthetic method is the same as that in Embodiment 1, only the substituents are different, and the target compound is obtained as beige white solid with a yield of 90.6%. mp=58.5-59.0° C. ¹H NMR (400 MHz, CDCl₃) δ 7.29 (d, J=4.8 Hz, thiophenyl-H, 1H), 7.01 (s, thiophenyl-H, 1H), 7.00-6.97 (m, thiophenyl-H, 1H), 5.58 (s, ═CH—, 1H), 4.71 (s, thiophenyl-CH₂—N, 2H), 3.10 (s, —CH₃, 3H). ¹³C NMR (101 MHz, CDCl₃) δ 189.42, 173.36, 136.90, 127.31, 127.26, 126.38, 93.66, 53.00, 39.53.

### Embodiment 26

### Synthesis of Compound I26 (R₁=2-chlorothiazoly; R₂=—CH₃)

The synthetic method is the same as that in Embodiment 1, only the substituents are different, and the target compound is obtained as a light yellow solid with a yield of 73%. mp=89.0-92.8° C. ¹H NMR (400 MHz, CDCl₃) δ 7.46 (s, 2-chlorothiazoly-H, 1H), 5.54 (s, ═CH—, 1H), 4.65 (d, J=1.2 Hz, Ph—CH₂—N, 2H), 3.08 (s, —CH₃, 3H). ¹³C NMR (101 MHz, CDCl₃) δ 189.23, 172.98, 153.02, 140.88, 133.97, 94.22, 50.49, 39.50.

Application embodiment 1: test of antibacterial activity of the compound

The antibacterial activity of the target compound against rice blast fungus is evaluated by mycelium growth rate method. 0.9 g of each tested compound is dissolved in 10 mL of DMF and diluted with 1000 Tween-80 water to obtain a solution with a concentration of 900 milligrams per milliliter (mg/mL). 5 mL of the obtained solution is added to 45 mL of sterile molten potato dextrose agar (PDA) to achieve the final test concentration (100 ppm). The PDA is then poured into sterile Petri dishes, with 15 mL being dispensed into each dish. For the blank control, 10 mL of 1% Tween-80 water sterile aqueous solution is mixed with 10 mL of DMF. Mycelial discs cut from subcultured PDA petri dishes are inoculated onto the PDA medium. The inoculated PDA petri dishes are incubated at 25° C. with 60% relative humidity for fungal growth. The growth inhibition rate is calculated according to standard methodology. Commercially available pydiflumetofen (100 ppm) is used as the positive control group, and its antibacterial activity is tested under identical conditions. Each treatment is performed in triplicate. The antibacterial activities are presented in Table 1.

TABLE 1
Antibacterial activity of synthetic compound I

| Compound | 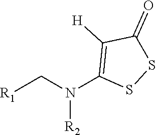 | | Antibacterial rate of rice blast fungus at 100 ppm |
|---|---|---|---|
| number | R₁ | R₂ | concentration (%) |
| I1 | 3-fluorophenyl | H | 58.3 |
| I2 | Phenyl group | H | 74.3 |
| I3 | 3-chlorophenyl | H | 79.1 |
| I4 | 3-cyanophenyl | H | 26.2 |
| I5 | 3,5-methoxyphenyl | H | 32.6 |
| I6 | 4,5-methoxyphenyl | H | 76.5 |
| I7 | 2,4-methoxyphenyl | H | 75.4 |
| I8 | 2,3-methoxyphenyl | H | 79.1 |
| I9 | 4-fluorophenyl | H | 65.2 |
| I10 | 3-methoxyphenyl | H | 72.2 |
| I11 | 5,6-fluorophenyl | H | 87.2 |
| I12 | 3,6-chlorophenyl | H | 70.1 |
| I13 | 4-aminomethylphenyl | H | 36.9 |
| I14 | 4-methoxyphenyl | H | 59.4 |
| I15 | 3-cyanophenyl | —CH₃ | 82.4 |
| I16 | Phenyl group | —CH₃ | 90.4 |
| I17 | 3-chlorophenyl | —CH₃ | 78.6 |
| I18 | 3-fluorophenyl | —CH₃ | 83.4 |
| I19 | 4-fluorophenyl | —CH₃ | 73.8 |
| I20 | 3-methoxyphenyl | —CH₃ | 96.8 |
| I21 | 4-methoxyphenyl | —CH₃ | 97.9 |
| I22 | 3,5-methoxyphenyl | —CH₃ | 72.2 |
| I23 | 3-fluorophenyl | —CH₂CH₃ | 95.3 |
| I24 | 3-fluoromethoxyphenyl | H | 31.4 |
| I25 | Thienyl | —CH₃ | 23.1 |
| I26 | 2-chlorothiazolyl | —CH₃ | 6.3 |
| Pydiflumetofe | — | — | 100 |

The above are only the optional embodiments of this disclosure, but the protection scope of this disclosure is not limited to this. Any change or replacement that may be easily thought of by a person familiar with this technical field within the technical scope disclosed in this disclosure should be included in the protection scope of this disclosure. Therefore, the protection scope of this disclosure should be based on the protection scope of the claims.

What is claimed is:

1. A dithiocyclopentenone derivative, wherein a chemical structural formula is shown in formula (I): 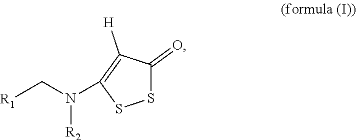
wherein R₁ is selected from phenyl or substituted phenyl, pyridyl, thiazolyl, thienyl or piperazinyl; and R₂ is selected from H, —CH₃ or —CH₂CH₃.

2. A method for preparing the dithiocyclopentenone derivative according to claim 1, comprising following steps:
stirring 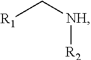
anhydrous potassium carbonate and tetrahydrofuran in an ice water bath, adding dichloro-1,2-dithiocyclopentenone, and reacting at room temperature to obtain an intermediate; and
dissolving the intermediate in acetonitrile, adding iodotrimethylsilane, reacting under a condition of nitrogen at room temperature, stopping the reaction when raw materials are completely reacted, continuously dropping saturated sodium bicarbonate solution and dichloromethane into reaction product for extraction, combining organic phases, and finally stripping once by sodium thiosulfate, drying, concentrating under reduced pressure, and performing column chromatography to obtain the dithiocyclopentenone derivative.

3. The method for preparing the dithiocyclopentenone derivative according to claim 2, wherein a molar ratio of the 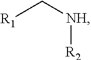
the anhydrous potassium carbonate and the dichloro-1,2-dithiocyclopentenone is 1:2:2; and
a dosage ratio of the 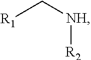
to the tetrahydrofuran is 1 g: 40 mL.

4. The method for preparing the dithiocyclopentenone derivative according to claim 2, wherein a molar ratio of the intermediate to the iodotrimethylsilane is 1:4; and
a dosage ratio of the intermediate to the acetonitrile is 0.3 g: 10 mL.

5. A bactericide, comprising 1-95 wt % of the dithiocyclopentenone derivative according to claim 1.
